(19) United States
(12) Patent Application Publication
(10) Pub. No.: US 2026/0174823 A1
(43) Pub. Date: Jun. 25, 2026

(54) CGRP/RAMP1 BLOCKADE TO TREAT ENDOMETRIOSIS-ASSOCIATED PAIN AND REDUCE ENDOMETRIOSIS LESIONS

(71) Applicant: The Children's Medical Center Corporation, Boston, MA (US)

(72) Inventors: Michael S. Rogers, Needham, MA (US); Victor Fattori, Brookline, MA (US)

(73) Assignee: The Children's Medical Center Corporation, Boston, MA (US)

(21) Appl. No.: 19/128,902

(22) PCT Filed: Nov. 9, 2023
(86) PCT No.: PCT/US2023/079238

§ 371 Date: May 9, 2025

Related U.S. Application Data
(60) Provisional application No. 63/424,472, filed on Nov. 10, 2022.

(51) Int. Cl. A61K 38/00; A61K 9/00

(52) U.S. Cl. CPC A61K 38/00; A61K 9/0019

(57) ABSTRACT

Provided herein are methods for treating endometriosis in a subject, said methods comprising the administration of an effective amount of an agent that inhibits CGRP/RAMP1 signaling to a subject in need thereof. Administration of an agent that inhibits CGRP/RAMP1 signaling may result in a reduction of pain associated with endometriosis in the subject, and/or a reduction of endometrial lesion size.

### CROSS-REFERENCE TO RELATED APPLICATIONS

This application claims the benefit under 35 U.S.C. § 119(e) of U.S. Provisional Application No. 63/424,472, filed Nov. 10, 2022, entitled “CGRP/RAMP1 BLOCKADE TO TREAT ENDOMETRIOSIS-ASSOCIATED PAIN AND REDUCE ENDOMETRIOSIS LESIONS”, the entire contents of which are incorporated herein by reference.

### BACKGROUND

Nociceptors are pain-sensing neurons that detect environmental cues and potentially damaging stimuli. However, aberrant activation of nociceptors is associated with the pathogenesis of pain-related disorders such as endometriosis, a painful inflammatory disease that affects up to 10% of individuals globally. Endometriosis is characterized by the development of endometrial-like lesions outside the uterus, which can lead to spontaneous pain and chronic pelvic pain. Current treatments for endometrial pain are limited to non-steroidal anti-inflammatory drugs (NSAIDs), other analgesics, hormonal agents, and surgical removal of endometrial lesions. While some endometriosis patients experience reduced pain as a result of these treatments, many patients still experience pain, while approximately 15% of patients experience no relief as a result of medical intervention.

### SUMMARY

The present disclosure is based on the discovery that calcitonin gene-related peptide (CGRP)/receptor activity modifying protein 1 (RAMP1) signaling promotes spontaneous pain and lesion size during endometriosis. Accordingly, some aspects of the present disclosure relate to a method for treating endometriosis in a subject, the method comprising administering to a subject in need thereof an effective amount of an agent that is sufficient of inhibiting CGRP/RAMP1 signaling in the subject. Other aspects of the present disclosure provide methods of treating endometrioma, the method comprising administering to a subject in need thereof an effective amount of an agent that is sufficient to inhibit calcitonin gene-related peptide/receptor activity modifying protein 1 (CGRP/RAMP1) signaling in the subject.

In some embodiments, the agent comprises a small molecule or an antibody.

In some embodiments, the agent is a small molecule that binds to CGRP or RAMP1. In some embodiments, the small molecule specifically binds to CGRP or RAMP1. In some embodiments, the small molecule binds to CGRP or RAMP1 on the cell surface of endometrial cells and/or macrophages of the subject. In some embodiments, the small molecule inhibits the CGRP or RAMP1 signaling in endometrial cells and/or macrophages of the subject. In some embodiments, the small molecule inhibits the signaling of CGRP or RAMP1 in endometrial cells and/or macrophages of the subject by at least 10%, at least 20%, at least 30%, at least 40%, at least 50%, at least 60%, at least 70%, at least 80%, at least 90%, at least 95%, or at least 99%.

In some embodiments, the small molecule binds to RAMP1 is Rimegepant.

In some embodiments, the small molecule binds to RAMP1 is Ubrogepant.

In some embodiments, the agent is an antibody that binds to CGRP or RAMP1. In some embodiments, the antibody specifically binds to CGRP or RAMP1. In some embodiments, the antibody binds to CGRP or RAMP1 on the cell surface of endometrial cells and/or macrophages of the subject. In some embodiments, the antibody inhibits CGRP or RAMP1 signaling in endometrial cells and/or macrophages of the subject. CGRP released by nociceptors may act on RAMP1 expressed on the cell surface of endometrial cells and/or macrophages. In some embodiments, the antibody inhibits the signaling of CGRP or RAMP1 in endometrial cells and/or macrophages of the subject by at least 10%, at least 20%, at least 30%, at least 40%, at least 50%, at least 60%, at least 70%, at least 80%, at least 90%, at least 95%, or at least 99%.

In some embodiments, the antibody binds to CGRP and is Fremanezumab, Eptinezumab or Galcanezumab.

In some embodiments, the antibody binds to RAMP1 and is Erenumab.

In some embodiments, the method comprises administering an effective amount of the agent that is sufficient to inhibit CGRP/RAMP1 signaling in endometrial cells and/or macrophages of the subject. In some embodiments, the endometrial cells are located in endometrial lesions of the subject. In some embodiments, the macrophages are peritoneal macrophages or macrophages located in reproductive tissue of the subject.

In some embodiments, the method results in a reduction of inflammation in endometrial lesions of the subject. In some embodiments, the method results in a reduction of nociception in endometrial lesions of the subject. In some embodiments, the method results in an enhancement of efferocytosis in macrophages of the subject. In some embodiments, the macrophages are peritoneal macrophages or macrophages located in reproductive tissue of the subject.

In some embodiments, the subject is a human patient.

In some embodiments, the administration is oral or via injection. In some embodiments, the administration is systemic. In some embodiments, the administration is local. In some embodiments, the agent is administered to endometrial lesions of the subject. In some embodiments, the agent is administered more than once.

### BRIEF DESCRIPTION OF DRAWINGS

The accompanying drawings are not intended to be drawn to scale. In the drawings, each identical or nearly identical component that is illustrated in various FIGS. is represented by a like numeral. For purposes of clarity, not every component may be labeled in every drawing.

In the drawings:

FIGS. 1A-1C show that treatment with Rimegepant, a small molecule RAMP1 antagonist, reduces endometriosis-associated pain and lesion size. Treatment with vehicle or Rimegepant at different doses was performed starting at 29 dpi to 42 dpi (daily, per oral gavage). FIG. 1A shows a schematic depicting the experimental design for blockage of CGRP/RAMP1 in a murine model for endometriosis pain. FIG. 1B shows quantification of von Frey testing at baseline and at 7, 14, 21, 28, 35, and 42 days following endometriosis induction in mice treated with 0, 3, 10, or 30 mg/kg Rimegepant. Data indicate means (n=10 mice per group). Significance was determined by two-way repeated-measures ANOVA followed by Tukey's post hoc. “*” indicates significance as compared to 0 mg/kg. “#” indicates significance as compared to 10 mg/kg Rimegepant. */#: p<0.05; **/##: p<0.01; ***/###: p<0.001. FIG. 1C shows the frequency of abdominal squashing, the frequency of abdominal contortions, and lesion size (mm) in mice treated with 0, 3, 10, or 30 mg/kg Rimegepant.

FIGS. 2A-2C show that treatment with QX-314, a lidocaine charged analog that blocks activated TRPV1 nociceptors, reduces endometriosis-associated pain and lesion size. Treatment with vehicle or QX-314 at different doses was performed starting at 29 dpi to 35 dpi (daily, i.p.). FIG. 2A shows a schematic depicting the experimental design for blockage of activated TRPV1 nociceptors in a murine model for endometriosis pain. FIG. 2B shows quantification of von Frey testing at baseline and at 7, 28, and 35 days following endometriosis induction in mice treated with 0, 0.1, or 1 mg/kg QX-314. Data indicate means (n=10 mice per group). Significance was determined by two-way repeated-measures ANOVA followed by Tukey's post hoc. “*” indicates significance as compared to 0 mg/kg. “#” indicates significance as compared to 0.1 mg/kg QX-314. */#: p<0.05; **/##: p<0.01; ***/###: p<0.001. FIG. 2C shows the frequency of abdominal squashing, the frequency of abdominal contortions, and lesion size (mm) in mice treated with 0, 0.1, or 1 mg/kg QX-314.

FIGS. 3A-3C show that treatment with Ubrogevant, Fremanezumab, or Galcanezumab reduce endometriosis-associated pain and lesion size. Treatment with vehicle, Ubrogepant (Ubrelvy) at 50 mg/kg, Fremanezumab (Ajovy) at 100 mg/kg, or Galcanezumab (Emgality) at 4 mg/kg was performed starting at 29 dpi to 56 dpi (daily, per oral gavage (Ubrogevant) or subcutaneous injection (Fremanezumab, Galcanezumab)). FIG. 3A shows schematic depicting the experimental designs for blockage of CGRP/RAMP1 in a murine model for endometriosis pain. FIG. 3B shows quantification of von Frey testing at baseline and at 7, 14, 21, 28, 35, 42, 49, and 56 days following endometriosis induction in mice treated with Ubrogevant, Fremanezumab, or Galcanezumab. FIG. 3C shows the frequency of abdominal squashing, the frequency of abdominal contortions, and lesion size (mm) in mice treated with Ubrogevant, Fremanezumab, or Galcanezumab.

FIGS. 4A-4C show treatment with Rimegepant. FIG. 4A shows the quantification of the number of mouse endometrial epithelial cells (mEECs) cultured in the presence macrophages, CGRP stimulation, and/or Rimegepant (n=8 per group). Peritoneal macrophages were plated on cell culture insert of transwells, while mEECs were plated on the bottom of each well. Addition of Rimegepant in the presence of CGRP stimulation reduces mEEC proliferation that is observed during co-culture with peritoneal macrophages. Error bars represent SEM. Significance was calculated by one-way ANOVA followed by Tukey's post hoc.: p<0.0001. FIG. 4B shows flow cytometry of 12z-eGFP immortalized human endometriotic cells that have been stimulated with CGRP and treated with etoposide to induce apoptosis. FIG. 4C shows quantification of efferocytosis as determined from eGFP fluorescence observed in macrophages to which apoptotic 12z-eGFP cells were added. Although CGRP stimulation reduces efferocytosis in macrophages, addition of Rimegepant restores normal levels of efferocytosis. Error bars represent SEM. Significance was calculated by one-way ANOVA followed by Tukey's post hoc. *: p<0.05; **: p<0.01.

FIGS. 5A-5C show that human and mouse endometriosis lesions express CGRP+fibers and endometriosis activates CGRP+ DRG neurons.

FIGS. 6A-6F show that endometriosis lesion activates TRPV1+ and TRPA1+ nociceptors and induce CGRP release.

FIGS. 7A-7N show that TRPV1+ nociceptors control endometriosis lesions establishment and growth.

FIGS. 8A-8D show that TRPV1+ nociceptors control F4/80+Ly6C+ immune cell recruitment during endometriosis.

FIGS. 9A-9D show that CGRP/RAMP1 signaling on macrophages support endometriosis lesion growth.

### DETAILED DESCRIPTION OF CERTAIN EMBODIMENTS

Aspects of the present disclosure are based on the discovery that calcitonin gene-related peptide (CGRP)/receptor activity modifying protein 1 (RAMPβ1) signaling promotes spontaneous pain and lesion size during endometriosis. Without wishing to be bound by theory, peptidergic nociceptors, such as those that release CGRP in response to nociception, are critically involved in the pain sensed during endometriosis. Furthermore, CGRP/RAMP1 signaling drives inflammation, lesion size, and reduces the activity of macrophages that typically function to clear dead cells from endometrial and other reproductive tissues. In view of this discovery and as exemplified herein, agents that antagonize (inhibit) CGRP/RAMP1 signaling may be utilized to inhibit nociception and/or reduce lesion size in a subject with endometriosis.

### Administration of Agents for Inhibiting CGRP/RAMPI Signaling

The present disclosure provides methods for the administration of an agent that is sufficient to inhibit CGRP/RAMP1 signaling, or a composition thereof (e.g., a pharmaceutical composition), to a subject. In some embodiments, a method is provided for treating or preventing a disease in a subject by administering an agent that is sufficient to inhibit CGRP/RAMP1 signaling, or a composition thereof (e.g., a pharmaceutical composition), to a subject. In some embodiments, a therapeutically effective amount of the agent is administered to the subject such that the method results in the treatment or prevention of a disease in the subject. In some embodiments, the disease is endometriosis. In some embodiments, the disease is treatment-resistant endometriosis, for which endometrial pain has not been reduced as a result of other treatments previously.

As used herein, the term “CGRP” refers to any peptide expressed from a gene encoding CGRP, e.g., α-CGRP (calcitonin related polypeptide alpha (CALCA)) or β-CGRP (calcitonin related polypeptide beta (CALCB)), each of which are expressed from a different genetic locus within the Homo sapiens genome. In some embodiments, a CGRP peptide contemplated herein is α-CGRP (NCBI Reference Sequence: NP_001732.1; Gene ID: 796). In some embodiments, a CGRP peptide contemplated herein is β-CGRP (NCBI Reference Sequence: NP_000719.1; Gene ID: 797). In some embodiments, a CGRP peptide contemplated herein is a CGRP peptide (e.g., α-CGRP, β-CGRP) that is endogenously expressed by a subject, e.g., a subject having endometriosis. In some embodiments, a CGRP peptide contemplated herein is a CGRP peptide (e.g., α-CGRP, β-CGRP) that is endogenously expressed by a human subject. In some embodiments, a CGRP peptide contemplated herein is a CGRP peptide endogenously expressed by a non-human subject, such as a non-human mammal, which is homologous to human CGRP (e.g., α-CGRP, β-CGRP).

As used herein, the term “RAMP1” refers to any protein expressed from a gene encoding RAMP1. In some embodiments, a RAMP1 protein contemplated herein is Homo sapiens RAMP1 (NCBI Reference Sequence: NP_005846.1; NCBI Reference Sequence: NP_001295282.1; Gene ID: 10267). In some embodiments, a RAMP1 protein contemplated herein is a RAMP1 protein that is endogenously expressed by a subject, e.g., a subject having endometriosis. In some embodiments, a RAMP1 protein contemplated herein is a RAMP1protein that is endogenously expressed by a human subject. In some embodiments, a RAMP1protein contemplated herein is RAMP1 protein endogenously expressed by a non-human subject, such as a non-human mammal, which is homologous to human RAMP1.

“Inhibit,” as used herein, means to reduce the level of CGRP/RAMP1 signaling. For example, an agent that reduces the level of CGRP/RAMP1 signaling may reduce the level of CGRP/RAMP 1 signaling by at least 30% (e.g., by at least 30%, at least 40%, at least 50%, at least 60%, at least 70%, at least 80%, at least 90%, at least 95%, at least 99%, or more), compared to in the absence of the agent.

“Endometriosis” refers to a condition in which endometrium-like tissue grows in locations outside the uterus. When endometriosis occurs in the ovary, endometriotic cells can form a menstrual fluid-filled sac, which is referred to as “endometrioma.”

As used herein, the terms “administer,” “administering,” or “administration” refer to implanting, absorbing, ingesting, injecting, inhaling, or otherwise introducing an agent described herein, or a composition thereof (e.g., a pharmaceutical composition), in or on a subject. As used herein, the term “treatment,” “treat,” and “treating” refers to the application or administration of an agent described herein, or a composition thereof (e.g., a pharmaceutical composition), to a subject in need thereof for the purpose of reducing the severity of a disease (e.g., endometriosis) in the subject. A “subject in need thereof” refers to an individual that has a disease, a symptom of the disease, or a predisposition toward the disease. A method for treating a disease may encompass administering to a subject an agent described herein, or a composition thereof (e.g., a pharmaceutical composition) with the intention to cure, heal, alleviate, relieve, alter, remedy, ameliorate, improve, or affect the disease, a symptom of the disease, or predisposition toward the disease in the subject. A method for treating a disease may encompass prophylaxis, wherein an agent is administered to the subject for the purpose of preventing development of the disease, for example, in a subject that is not known to have the disease, but may develop or be at risk of developing the disease in the future.

As used herein, a “therapeutically effective amount” or “effective amount” refers to the amount of an agent (e.g., an agent described herein) that is sufficient to elicit the desired biological response in the subject, for example, alleviating one or more symptoms of the disease (e.g., endometriosis). A therapeutically effective amount may be an amount that is either administered to the subject alone or in combination with one or more other agents. Effective amounts vary, as recognized by those skilled in the art, depending on such factors as the desired biological endpoint, the pharmacokinetics of the administered agent, the particular condition or disease being treated, the severity of the condition or disease, the individual parameters of the subject, including age, physical condition, size, gender and weight, the duration of the treatment, the nature of any other concurrent therapy, the specific route of administration, and like factors that are within the knowledge and expertise of the health practitioner to determine. These factors are well known to those of ordinary skill in the art and can be addressed with no more than routine experimentation. It is generally preferred that a maximum dose of the individual agents described herein (e.g., an agent described herein) or any combinations thereof to be used is at most the highest dose that can be safely administered to the subject according to sound medical judgment. Preferably, an effective dose is lower than the highest dose that can be safely administered to the subject. It will be understood by those of ordinary skill in the art, however, that a subject or health practitioner may select a lower dose (e.g., the minimum effective dose) in order to mitigate any potential risks of treatment, such as side effects of the treatment.

In some embodiments, for an adult subject of normal weight, doses ranging from about 0.01 to 1000 mg/kg of an agent (e.g., an agent described herein) may be administered. In some embodiments, the dose is between 1 to 200 mg. The particular dosage regimen, i.e., the dose, timing, and repetition, will depend on the particular subject and that subject's medical history, as well as the properties of the agent (such as the pharmacokinetics of the agent) and other consideration well known in the art.

Treating a disease (e.g., endometriosis or endometrioma) may include delaying the development or progression of the disease or reducing disease severity. Treating the disease does not necessarily require curative results. As used herein, “delaying” the development of a disease means to defer, hinder, slow, retard, stabilize, and/or postpone progression of the disease in a subject. Delaying the progression of a disease may include delaying or preventing the spread of a disease occurring in a subject, such as, for example, delaying or preventing the spread of endometriosis occurring in a subject (i.e., endometrial lesions) to tissues not yet affected by endometriosis. This delay can be of varying lengths of time, depending on the history of the disease and/or individuals being treated. A method that delays the development of a disease, or delays the onset of the disease, is a method that reduces probability of developing one or more symptoms of the disease in a given time frame and/or reduces extent of the symptoms in a given time frame, as compared to the absence of such a method. Comparisons are typically based on clinical studies, using a number of subjects sufficient to give a statistically significant result.

The “development” or “progression” of a disease (e.g., endometriosis or endometrioma) refers to initial manifestations and/or ensuing progression of the disease in a subject.

Development of a disease can be detectable and assessed using standard clinical techniques as well known in the art. However, development also refers to progression that may be undetectable. For purpose of this disclosure, development or progression may refer to the development or progression of symptoms of a disease. The term “development” includes the occurrence, recurrence, and onset of a disease. As used herein “onset” or “occurrence” of a disease includes the initial onset of a disease, as well as recurrence of the disease (i.e., in a subject who has had the disease previously).

In some embodiments, the agent with which a subject is treated comprises a small molecule or an antibody.

In some embodiments, the agent is an antibody (e.g., a monoclonal antibody) that binds to CGRP or RAMP1. In some embodiments, the antibody preferentially binds to CGRP or RAMP1 (i.e., the antibody may also bind to other species, but with lower affinity as compared to CGRP or RAMP1). In some embodiments, the antibody specifically binds to CGRP or RAMP1.In some embodiments, the antibody binds to CGRP or RAMP1 on the cell surface of endometrial cells and/or macrophages of the subject. In some embodiments, binding of the antibody to CGRP or RAMP1 (e.g., on the surface of endometrial cells or macrophages of the subject) results in inhibition of CGRP or RAMP1 (e.g., in endometrial cells or macrophages of the subject). In some embodiments, binding between the antibody and CGRP or RAMP1 results in inhibition of the activity of CGRP or RAMP1 (e.g., CGRP/RAMP1 signaling activity) in endometrial cells and/or macrophages of the subject by at least 10%, at least 20%, at least 30%, at least 40%, at least 50%, at least 60%, at least 70%, at least 80%, at least 90%, at least 95%, or at least 99%.

In some embodiments, the agent is an antibody (e.g., a monoclonal antibody) that binds to CGRP or RAMP1 and is generally known among those of ordinary skill in the relevant art (e.g., for the purpose of treating migraine). For example, in some embodiments, an antibody binding to CGRP is Fremanezumab (AJOVY®) or Galcanezumab (EMGALITY®) or Eptinezumab. In some embodiments, an antibody binding to RAMP1 is Erenumab (AIMOVIG®).

An “antibody” or “immunoglobulin (Ig)” is a large, Y-shaped protein produced mainly by plasma cells that is used by the immune system to neutralize an exogenous substance (e.g., a pathogens such as bacteria and viruses). Antibodies are classified as IgA, IgD, IgE, IgG, and IgM. “Antibodies” and “antibody fragments” include whole antibodies and any antigen binding fragment (i.e., “antigen-binding portion”) or single chain thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the a and y chains and four CH domains for u and & isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference).

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and α classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

An “antibody fragment” for use in accordance with the present disclosure contains the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, an antibody fragment may be a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and-binding site. This fragment consists of a dimer of one heavy-and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

Single-chain Fv also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

Antibodies may be isolated. An isolated antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

In some embodiments, the antibody of the present disclosure is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibody of the present disclosure is a polyclonal antibody. A “polyclonal antibody” a mixture of different antibody molecules which react with more than one immunogenic determinant of an antigen. Polyclonal antibodies may be isolated or purified from mammalian blood, secretions, or other fluids, or from eggs. Polyclonal antibodies may also be recombinant. A recombinant polyclonal antibody is a polyclonal antibody generated by the use of recombinant technologies. Recombinantly generated polyclonal antibodies usually contain a high concentration of different antibody molecules, all or a majority of (e.g., more than 80%, more than 85%, more than 90%, more than 95%, more than 99%, or more) which are displaying a desired binding activity towards an antigen composed of more than one epitope.

Methods of producing antibodies (e.g., monoclonal antibodies or polyclonal antibodies) are known in the art. For example, a polyclonal antibody may be prepared by immunizing an animal, preferably a mammal, with an allergen of choice followed by the isolation of antibody-producing B-lymphocytes from blood, bone marrow, lymph nodes, or spleen. Alternatively, antibody-producing cells may be isolated from an animal and exposed to an allergen in vitro against which antibodies are to be raised. The antibody-producing cells may then be cultured to obtain a population of antibody-producing cells, optionally after fusion to an immortalized cell line such as a myeloma. In some embodiments, as a starting material B-lymphocytes may be isolated from the tissue of an allergic patient, in order to generate fully human polyclonal antibodies. Antibodies may be produced in mice, rats, pigs (swine), sheep, bovine material, or other animals transgenic for the human immunoglobulin genes, as starting material in order to generate fully human polyclonal antibodies. In some embodiments, mice or other animals transgenic for the human immunoglobulin genes (e.g., as disclosed in U.S. Pat. No. 5,939,598), the animals may be immunized to stimulate the in vivo generation of specific antibodies and antibody producing cells before preparation of the polyclonal antibodies from the animal by extraction of B lymphocytes or purification of polyclonal serum.

Monoclonal antibodies are typically made by cell culture that involves fusing myeloma cells with mouse spleen cells immunized with the desired antigen (i.e., hyrbidoma technology). The mixture of cells is diluted and clones are grown from single parent cells on microtitre wells. The antibodies secreted by the different clones are then assayed for their ability to bind to the antigen (with a test such as ELISA or Antigen Microarray Assay) or immuno-dot blot. The most productive and stable clone is then selected for future use.

In some embodiments, the antibodies described herein are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, the agent is a small molecule that binds to CGRP or RAMP1. A “small molecule,” as used herein, refers to a molecule of low molecular weight (e.g., <900 daltons) organic or inorganic compound that may function in regulating a biological process. Nonlimiting examples of a small molecule include lipids, monosaccharides, second messengers, other natural products and metabolites, as well as drugs and other xenobiotics. A “lipid” refers to a group of naturally occurring molecules that include fats, waxes, sterols, fat-soluble vitamins (such as vitamins A, D, E, and K), monoglycerides, diglycerides, triglycerides, phospholipids, and others. A “monosaccharide” refers to a class of sugars (e.g., glucose) that cannot be hydrolyzed to give a simpler sugar. Non-limiting examples of monosaccharides include glucose (dextrose), fructose (levulose) and galactose. A “second messenger” is a molecule that relay signals received at receptors on the cell surface (e.g., from protein hormones, growth factors, etc.) to target molecules in the cytosol and/or nucleus. Nonlimiting examples of second messenger molecules include cyclic AMP, cyclic GMP, inositol trisphosphate, diacylglycerol, and calcium. A “metabolite” is an molecule that forms as an intermediate produce of metabolism. Non-limiting examples of a metabolite include ethanol, glutamic acid, aspartic acid, 5′guanylic acid, Isoascorbic acid, acetic acid, lactic acid, glycerol, and vitamin B2. A “xenobiotic” is a foreign chemical substance found within an organism that is not normally naturally produced by or expected to be present within. Non-limiting examples of xenobiotics include drugs, antibiotics, carcinogens, environmental pollutants, food additives, hydrocarbons, and pesticides.

In some embodiments, the small molecule preferentially binds to CGRP or RAMP1 (i.e., the small molecule may also bind to other species, but with lower affinity a s compared to CGRP or RAMP1). In some embodiments, the small molecule specifically binds to CGRP or RAMP1. In some embodiments, the small molecule binds to CGRP or RAMP1 on the cell surface of endometrial cells and/or macrophages of the subject. In some embodiments, binding of the small molecule to CGRP or RAMP1 (e.g., on the surface of endometrial cells or macrophages of the subject) results in inhibition of CGRP or RAMP1 (e.g., in endometrial cells or macrophages of the subject). In some embodiments, binding between the small molecule and CGRP or RAMP1 results in inhibition of the activity of CGRP or RAMP1 (e.g., CGRP/RAMP1 signaling activity) in endometrial cells and/or macrophages of the subject by at least 10%, at least 20%, at least 30%, at least 40%, at least 50%, at least 60%, at least 70%, at least 80%, at least 90%, at least 95%, or at least 99%.

In some embodiments, the agent is a small molecule that binds to CGRP or RAMP1 and is generally known among those of ordinary skill in the relevant art (e.g., for the purpose of treating migraine). For example, in some embodiments, a small molecule binding to RAMP1 is Rimegepant (NURTEC®) or Ubrogepant (UBRELVY®).

In some embodiments, a method described herein for treating endometriosis in a subject comprises administering an effective amount of an agent that is sufficient to inhibit CGRP/RAMP1 signaling in endometrial cells and/or macrophages of the subject. In some embodiments, the endometrial cells are located in endometrial lesions of the subject. In some embodiments, the macrophages are peritoneal macrophages or macrophages located in reproductive tissue of the subject.

In some embodiments, a method described herein for treating endometriosis in a subject results in a reduction of inflammation in endometrial lesions of the subject. In some embodiments, a method described herein results in a reduction of inflammation in endometrial lesions of the subject by at least 10%, at least 20%, at least 30%, at least 40%, at least 50%, at least 60%, at least 70%, at least 80%, at least 90%, at least 95%, at least 99%, or 100%. A person of ordinary skill in the art is sufficiently capable of assessing inflammation in endometrial lesions and may, for example, assess changes in the levels of inflammatory cytokines and/or chemokines present in endometrial lesions of the subject.

In some embodiments, a method described herein for treating endometriosis in a subject results in a reduction of nociception in endometrial lesions of the subject. In some embodiments, a method described herein results in a reduction of nociception in endometrial lesions of the subject by at least 10%, at least 20%, at least 30%, at least 40%, at least 50%, at least 60%, at least 70%, at least 80%, at least 90%, at least 95%, at least 99%, or 100%. A person of ordinary skill in the art is sufficiently capable of assessing inflammation in endometrial lesions and may, for example, qualitatively score endometrial pain (e.g., spontaneous endometrial pain) experienced by the subject following treatment, as compared to before treatment.

In some embodiments, a method described herein for treating endometriosis in a subject results in an enhancement of efferocytosis in macrophages of the subject. In some embodiments, a method described herein results in an enhancement of efferocytosis in macrophages of the subject by at least 10%, at least 20%, at least 30%, at least 40%, at least 50%, at least 60%, at least 70%, at least 80%, at least 90%, at least 95%, at least 99%, or 100%. In some embodiments, the macrophages are peritoneal macrophages or macrophages located in reproductive tissue (e.g., endometrial tissue, uterine tissue, fallopian tissue, ovarian tissue) of the subject. A person of ordinary skill in the art is sufficiently capable of assessing efferocytosis in macrophages of the subject and may, for example, obtain a sample containing macrophages from the subject, provide an efferocytotic substrate to the macrophages, and assess efferocytotic activity of the macrophages following treatment, as compared to before treatment.

A “subject” to which administration is contemplated herein may refer to a human (i.e., male or female of any age group, e.g., pediatric subject (e.g., infant, child, or adolescent) or adult subject (e.g., young adult, middle-aged adult, or senior adult)) or a non-human animal (e.g., a research animal, such as a rat or mouse, or a companion animal, such as a feline or canine). In some embodiments, the subject is a human patient (e.g., a human patient known to have or at risk for endometriosis).

Conventional methods, known to those of ordinary skill in the art of medicine, can be used to administer an agent described herein or a composition thereof (e.g., a pharmaceutical composition) to the subject, depending upon the type of disease (e.g., endometriosis) to be treated or the site of the disease (e.g., reproductive tract). An agent or composition thereof (e.g., a pharmaceutical composition) can be administered systemically (i.e., throughout the body) or locally (i.e., to one or more specific organs, tissues, or locations in the body). The agent or composition thereof (e.g., a pharmaceutical composition) can be administered via any conventional route, e.g., administered orally, parenterally, by inhalation spray, topically, rectally, nasally, buccally, vaginally, intraperitoneally, or via an implanted reservoir. The term “parenteral” as used herein includes subcutaneous, intracutaneous, intravenous, intramuscular, intraarticular, intraarterial, intraperitoneal, intralesional, and infusion techniques. In some embodiments, the agent or composition thereof (e.g., a pharmaceutical composition) is administered orally. In some embodiments, the agent or composition thereof (e.g., a pharmaceutical composition) is administered via intravenous injection or infusion. In addition, the agent or composition thereof (e.g., a pharmaceutical composition) can be administered to the subject via injectable depot routes of administration such as using 1-, 3-, or 6-month depot injectable or biodegradable materials and methods. In some embodiments, the agent or composition thereof (e.g., a pharmaceutical composition) is administered to reproductive tissue (e.g., endometrial tissue, uterine tissue, fallopian tissue, ovarian tissue) in the subject. In some embodiments, the agent or composition thereof (e.g., a pharmaceutical composition) is administered to an endometrial lesion occurring in the subject.

In some embodiments, the administration occurs more than once. In some embodiments, the administration occurs once per day, once per 2 days, once per 3 days, once per 4 days, once per 5 days, once per 6 days, once per week, once per 2 weeks, once per 3 weeks, once per month, once per 2 months, once per 3 months, once per 4 months, once per 6 months, once per 7 months, once per 8 months, once per 9 months, once per 10 months, once per 11 months, or once per year.

The agent that inhibits CGRP/RAMP1 signaling may be formulated in a pharmaceutical composition. In some embodiments, the pharmaceutical composition further comprises a pharmaceutical acceptable carrier. The term “pharmaceutically-acceptable carrier” as used herein means one or more compatible solid or liquid filler, diluents or encapsulating substances which are suitable for administration into a subject, e.g., a human. A pharmaceutically acceptable carrier is “acceptable” in the sense of being compatible with the other ingredients of the formulation and not injurious to the tissue of the patient (e.g., physiologically compatible, sterile, physiologic pH, etc.). The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation.

The pharmaceutical compositions may conveniently be presented in unit dosage form and may be prepared by any of the methods well-known in the art of pharmacy. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

The formulation of the pharmaceutical composition may dependent upon the route of administration. Injectable preparations suitable for parenteral administration or intratumoral, peritumoral, intralesional or perilesional administration include, for example, sterile injectable aqueous or oleaginous suspensions and may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3 propanediol or 1,3 butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

For topical administration, the pharmaceutical composition can be formulated into ointments, salves, gels, or creams, as is generally known in the art. Topical administration can utilize transdermal delivery systems well known in the art. An example is a dermal patch.

Compositions suitable for oral administration may be presented as discrete units, such as capsules, tablets, lozenges, each containing a predetermined amount of the anti-inflammatory agent. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the anti-inflammatory agent is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the pharmaceutical compositions used for therapeutic administration must be sterile. Sterility is readily accomplished by filtration through sterile filtration membranes (e.g., 0.2 micron membranes). Alternatively, preservatives can be used to prevent the growth or action of microorganisms. Various preservatives are well known and include, for example, phenol and ascorbic acid. The cyclic Psap peptide and/or the pharmaceutical composition ordinarily will be stored in lyophilized form or as an aqueous solution if it is highly stable to thermal and oxidative denaturation. The pH of the preparations typically will be about from 6 to 8, although higher or lower pH values can also be appropriate in certain instances.

### EXAMPLES

### Example 1: Nociceptor-Macrophage Crosstalk Drives Endometriosis Pain and Lesion Growth Introduction

Endometriosis is a painful inflammatory disease that affects up to 10% of individuals around the world with annual health care costs approaching $70 billion in the United States alone. Because of debilitating chronic pain, affected patients lose around 11 hours of work weekly due to reduced productivity. Current treatments for pain in endometriosis are limited to non-steroidal anti-inflammatory drugs (NSAIDs), other analgesics, hormonal agents, and surgical removal of the lesions (see, e.g., Becker et al., “Reevaluating response and failure of medical treatment of endometriosis: a systematic review.” Fertil Steril. 2017; 108(1): 125-136; Rizk et al., “Recurrence of endometriosis after hysterectomy.” Facts Views Vis Obgyn. 2014; 6(4): 219-27; and Stratton & Berkley. “Chronic pelvic pain and endometriosis: translational evidence of the relationship and implications.” Hum Reprod Update. 2011; 17(3): 327-46). While effective for a fraction of patients, hormonal therapies and NSAIDs present several side effects, and should be used with cautious by patients with comorbidities. Moreover, even after surgery, disease and pain recurrence are very common, ˜15% of individuals experience no relief with medical therapy, and up ˜30% experience recurrence of pain symptoms after treatment cessation. Therefore, new medical therapies and targets that provide long-term benefit are still required.

Nociceptors (pain-sensing neurons) detect environmental cues and potentially damaging stimuli, such as pathogens or injured tissue. Upon activation, a subpopulation of nociceptors, known as peptidergic nociceptors, release neuropeptides such as calcitonin gene-related peptide (CGRP) that signals through the receptor activity modifying protein 1 (RAMP1) to orchestrate inflammation in a context-dependent manner. High-doses of CGRP have previously been shown to increase the growth of endometriosis lesions, but whether blocking nociceptor-derived CGRP signaling would decrease pain or lesion size in the context of endometriosis was unclear.

Therefore, to test this hypothesis nociceptors were ablated in lesion-bearing mice either genetically, using TRPV1-cre-recombinase (cre) Diphtheria Toxin Fragment A (DTA) mice or resiniferatoxin toxin (RTX), and the effect of these treatments was assessed in this non-surgical mouse model of endometriosis (see Fattori et al., “Nonsurgical mouse model of endometriosis-associated pain that responds to clinically active drugs.” Pain. 2020; 161(6): 1321-1331). This model recapitulates not only mechanical abdominal pain but also spontaneous pain, which is the primary symptom of patients with chronic pain, including endometriosis. Similar to humans, the lesions in this model have been shown to be highly innervated with CGRP-positive fibers and presents glands, stromal and immune cells. Therefore, this model can be used to screen novel or reproposed drugs for endometriosis treatment.

### Methods

### Endometriosis Induction

After at least one week of acclimatization, donor mice received a subcutaneous injection of 3 μg/mouse estradiol benzoate to stimulate the growth of the endometrium. Seven days later, the uteri of the donor mice were dissected into a Petri dish containing HBSS and split longitudinally with a pair of scissors. Uterine horns from each donor mouse were minced with scissors and scalpel one at the time, ensuring that the maximal diameter of each fragment was consistently smaller than 1 millimeter (mm). Each dissociated uterine horn was then injected intraperitoneally using an 18 G needle into a recipient mouse in 500 μL of HBSS. One donor mouse was used for every two endometriosis mice.

### Von Frey Testing

For mechanical and heat hyperalgesia tests, mice were allowed to habituate to the apparatus for at least 2 hours and during three consecutive days before the beginning of measurements. After habituation, baseline measurements were obtained on two consecutive days prior to the induction of endometriosis. Pain intensity to a mechanical stimulus (mechanical hyperalgesia) in the abdominal region was measured using von Frey filaments. The experimenter was trained, and care was taken not to stimulate the same point consecutively, and the stimulation of the external genitalia was avoided. A jump or paw flinch was considered a withdrawal response. The mechanical threshold was determined by the up and down method starting with 0.4 g filament and calculated using the open-source software Up-Down Reader.

### Spontaneous Pain Quantification

For spontaneous abdominal pain measurements, stretching the abdomen (abdominal contortions) and squashing of the lower abdomen against the floor were quantified. Briefly, for the abdominal contortions, mice were placed in individual chambers in a temperature-controlled (29° C.) glass plate and the number of abdominal contortions was quantified for 10 minutes.

Positive responses consist of a contraction of the abdominal muscle together with stretching of hind limbs. For abdominal squashing, the number of times the mice pressed the lower abdominal region against the floor in five minutes was quantified.

### Lesion Size

Lesions were carefully dissected at 42 days post induction (dpi) and measured using calipers. Lesion size is expressed in millimeters (mm) calculated from the mean of two measurements (width and height).

### Peritoneal Macrophage Culture

Peritoneal cells were collected in FACS buffer from the abdominal cavity of naïve mice. Peritoneal cavity immune cells were seeded in petri dishes in RPMI media and incubated at 37° C. in 5% CO₂ for 60 minutes. Non-adherent cells were discarded, and adherent cells (macrophages) were scraped, centrifuged, and counted.

### Proliferation Assay

For experiments involving mouse epithelial endometrial cell (mEEC) proliferation, macrophages were plated (100,000 cells per well) onto tissue culture insert and left overnight with 20,000 mEEC plated on the bottom of the well. Stimulus with CGRP (100 nM) or vehicle was performed for 24 hours and then mEEC were used to determine for cell proliferation assay using CyQuant™ kit (Thermo Fisher Scientific #C7026). Treatment with Rimegepant (100 nM) or vehicle was performed 30 minutes before stimulus with CGRP.

For the efferocytosis assay, macrophages (200,000 cells per well) were plated overnight onto a 96-well plate. Stimulus with CGRP (100 nM) or vehicle was performed for 24 hours. After that, etoposide-induced apoptotic 12z-eGFP were added (600,000 cells per well) to the macrophage culture and incubated for 1 hour. Cells were washed 3 times with DPBS to remove non-engulfed 12z-eGFP and then and fluorescence intensity determined. Treatment with Rimegepant (100 nM) or vehicle was performed 30 minutes before stimulus with CGRP.

### Results

In both TRPV1-cre DTA and RTX-treated mice, it was observed that nociceptor ablation reduced lesion size, evoked pain, and spontaneous pain behaviors. The latter is of particular importance because spontaneous pain is the most problematic symptom in women with poorly-treated endometriosis. Based on these data, it was hypothesized that blocking CGRP signaling with either anti-CGRP antibodies or receptor antagonists (e.g., a small molecule or an antibody) would reduce endometriosis associated pain and lesion size.

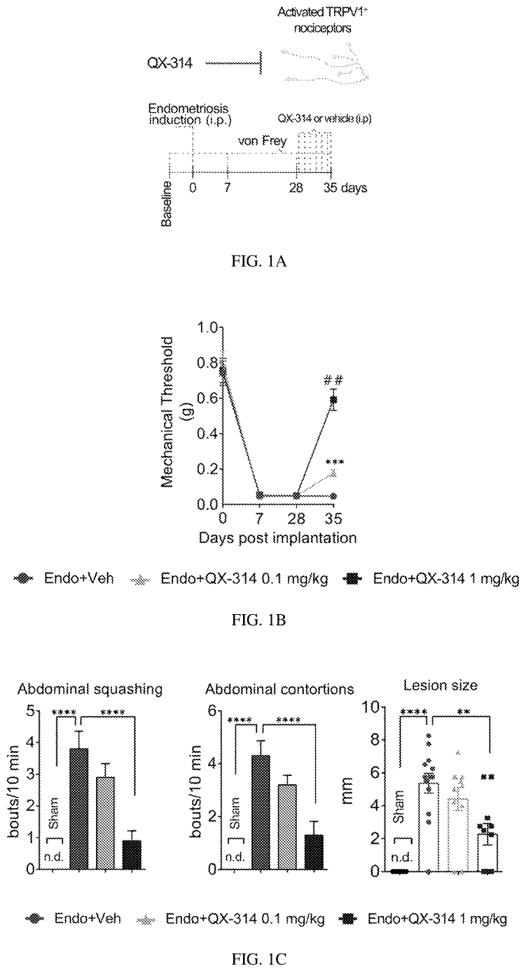

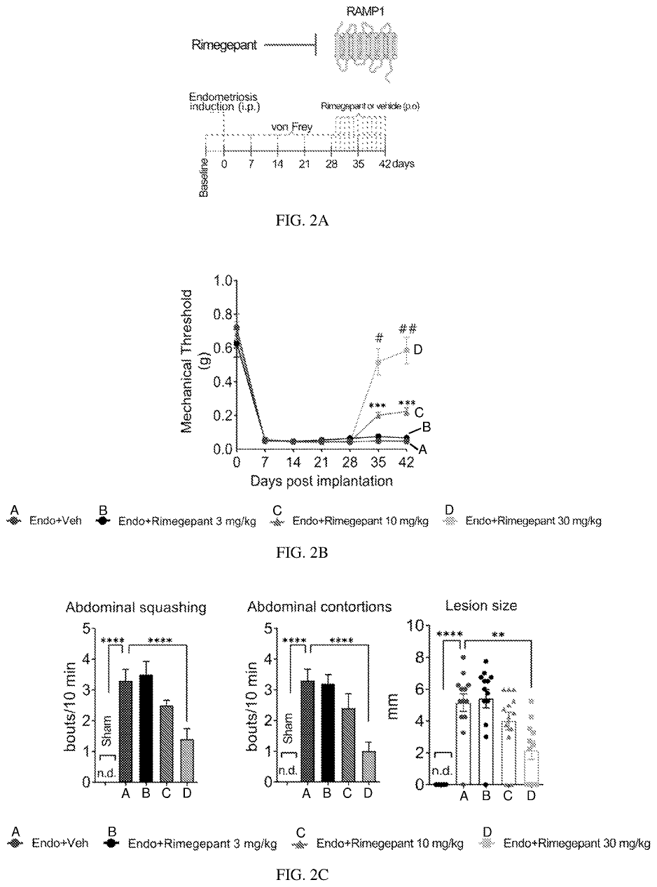

Rimegepant is a RAMP1 antagonist, which is currently used to treat as well as prevent migraine attacks (see, e.g., Edvinsson et al., “CGRP as the target of new migraine therapies successful translation from bench to clinic.” Nat Rev Neurol. 2018; 14(6): 338-350). Rimegepant shows a safe preclinical profile upon chronic treatment (26 weeks) and no adverse effects on fertility even at doses higher as 60 mg/kg. It was therefore tested whether Rimegepant is effective at reducing pain in a murine model (FIG. 1A). Endometriosis lesions were induced in 40 mice and at day 29 post-induction, mice were randomly assigned to treatment with vehicle or Rimegepant at 3, 10, or 30 mg/kg (n=10 mice per group). Treatment was performed daily by oral gavage. Mechanical abdominal pain was determined weekly, 7 to 42 days post implantation (dpi) using von Frey filaments, while abdominal squashing and contortions were determined by counting these pain behaviors. Lesion size was calculated from the mean of two measurements (width and height in mm). These methods were in accordance with previously studies (see Fattori et al., “Nonsurgical mouse model of endometriosis-associated pain that responds to clinically active drugs.” Pain. 2020; 161(6):1321-1331). It was observed that treatment with Rimegepant increased the abdominal mechanical threshold to the von Frey fibers, indicating reduction in mechanical pain (FIG. 1B), as well as reduced the number of abdominal squashing and contortion (FIG. 1C). It was also determined that Rimegepant reduced lesion size. These data demonstrate that blocking CGRP/RAMP1 signaling with Rimegepant reduces endometriosis-associated pain as well as reduces lesion size. Encouragingly, similar findings were also obtained in parallel experiments performed with mice treated with QX-314, a lidocaine charged analog that directly blocks activated TRPV1 nociceptors (FIG. 2A). As expected, mice treat treated with QX-314 also demonstrated reduced endometriosis-associated pain and reduced lesion size (FIGS. 2B and 2C).

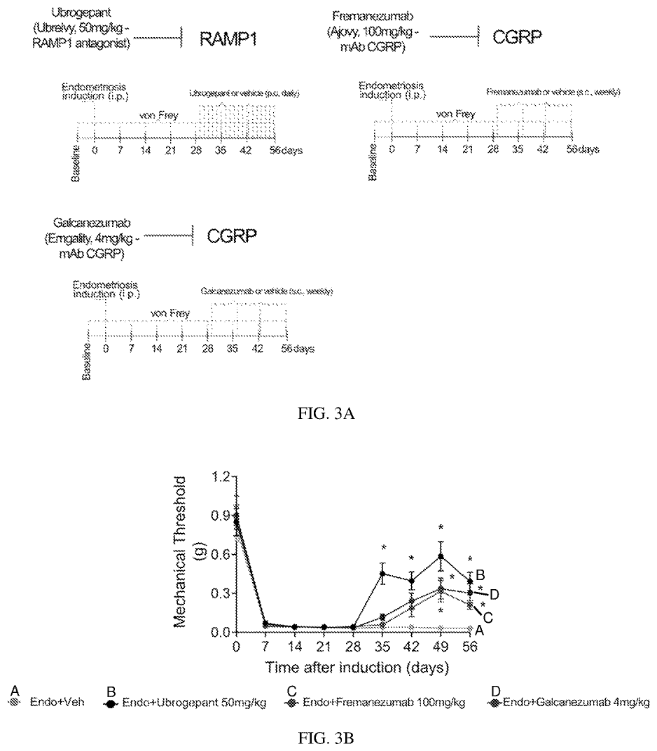

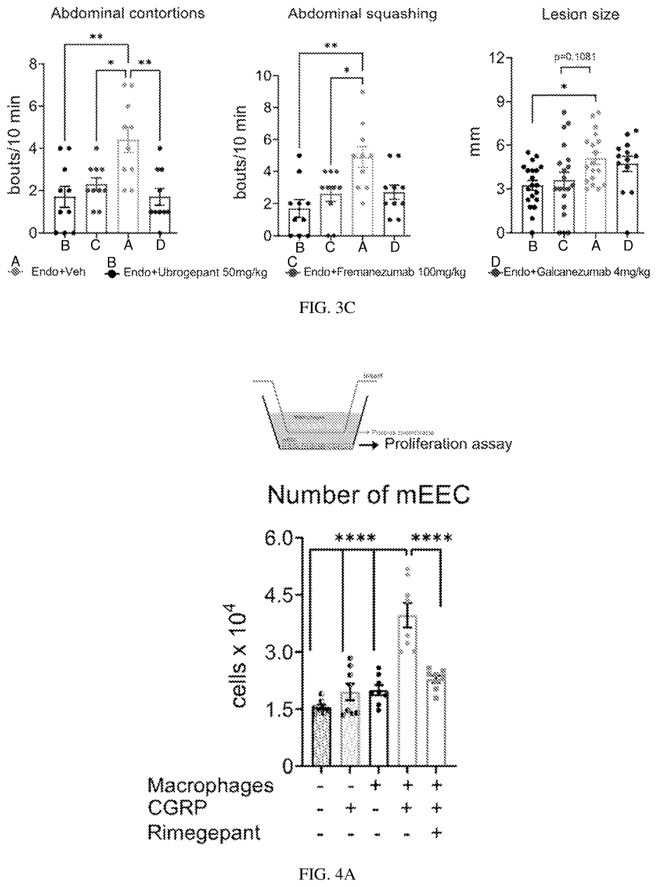

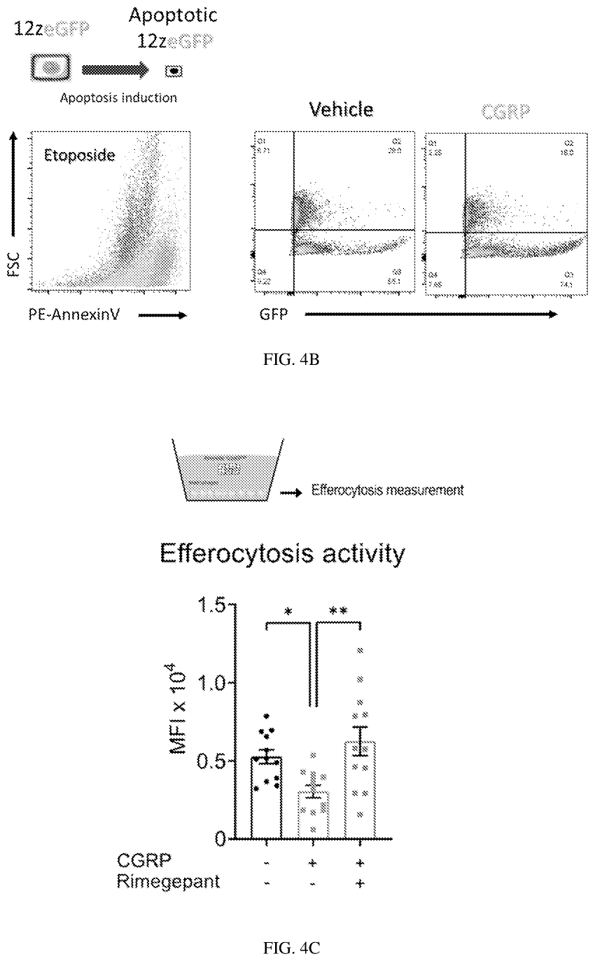

To further explore the effect of CGRP or RAMP1 antagonists on endometriosis-associated pain and lesion size, further studies were conducted with mice treated with Ubrogepant (an alternate RAMP1 antagonist to Rimegepant), Fremanezumab (a monoclonal antibody antagonist of CGRP), or Galcanezumab (a second monoclonal antibody antagonist of CGRP) (FIG. 3A). As observed in mice treated with Rimegepant, treatment with each of these CGRP/RAMP1 antagonists led to a significant reduction in endometriosis-associated pain (FIGS. 3B and 3C). Treatment with Ubrogepant and Fremanezumab were further associated with significantly smaller lesion sizes. These data confirm that the measurable benefits that are observed with Rimegepant treatment are not unique to that small molecule, and that conceivably any antagonistic agent that impedes CGRP/RAMP1 function could be used to reduce pain and/or lesion size during endometriosis.

Macrophages represent the majority of cells in the peritoneal cavity and these cells are directly implicated in the etiology of endometriosis. Depletion of macrophages in surgical-induced mouse models of endometriosis has been shown to reduce lesion weight, size, and vascularity (see, e.g., Ono et al., “CD206+ macrophage is an accelerator of endometriotic-like lesion via promoting angiogenesis in the endometriosis mouse model.” Sci Rep. 2021 13; 11(1):853; Duan et al., “The M2a macrophage subset may be critically involved in the fibrogenesis of endometriosis in mice.” Reprod Biomed Online. 2018; 37(3): 254-268). However, little is known about CGRP/RAMP1 signaling during endometriosis and how this might affect macrophage phenotype. Therefore, a co-culture assay was performed to address whether CGRP promotes the growth of endometriosis lesion. In this experiment, peritoneal macrophages were plated on a cell culture insert and co-cultured with a mouse epithelial endometrial cell line (mEEC) in a 24-well plate. Cell growth was assessed using the CyQuant™ kit and quantification performed in a spectrophotometer (n=8 well per group). CGRP stimulated macrophages were demonstrated to release mediators that promote the growth of mEEC, which was inhibited by treatment with Rimegepant. This outcome indicates that CGRP/RAMP1 signaling on macrophages is important to support mEEC growth. To determine whether CGRP could impair the efferocytosis activity of macrophages, a co-culture assay was again performed with peritoneal macrophages and apoptotic 12z-eGFP, an immortalized human endometriotic cell line that is tagged with GFP. Apoptosis of 12z-eGFP was induced with etoposide. It was observed that CGRP-stimulated macrophages display reduced efferocytosis activity as compared to the vehicle-treated group, as determined by reduced fluorescence intensity. This effect was rescued by Rimegepant, indicating that CGRP/RAMP1 signaling on macrophages impairs efferocytosis activity. Altogether, these data indicate that CGRP changes the phenotype of macrophages to a “pro-endometriosis phenotype,” thereby supporting cell growth and impairing efferocytosis of dead cells.

Altogether, these findings provide a proof-of-concept that blocking CGRP/RAMP1 signaling by using a clinically active RAMP1 antagonist (e.g., Rimegepant) is an effective strategy for reducing endometriosis-associated pain and lesion growth. These findings also demonstrate the importance of CGRP/RAMP1 on macrophages during endometriosis and indicate that Rimegepant is able to rescue the deleterious effects of CGRP. Based on these data, it is expected that other CGRP-or RAMP1-targeting drugs (e.g., antibodies or small molecules targeting CGRP/RAMP1 activity) will be also effective at reducing endometriosis burden. Therefore, blocking neuroimmune communication by disrupting CGRP/RAMP1 signaling could be explored as a non-hormonal and non-opioid approach for endometriosis treatment, and could provide an additional therapeutic option for patients whose endometriosis pain is not addressed by current therapies.

### Example 2: Human and Mouse Endometriosis Lesions Express CGRP+ Fibers and Endometriosis Activates CGRP+ DRG Neurons

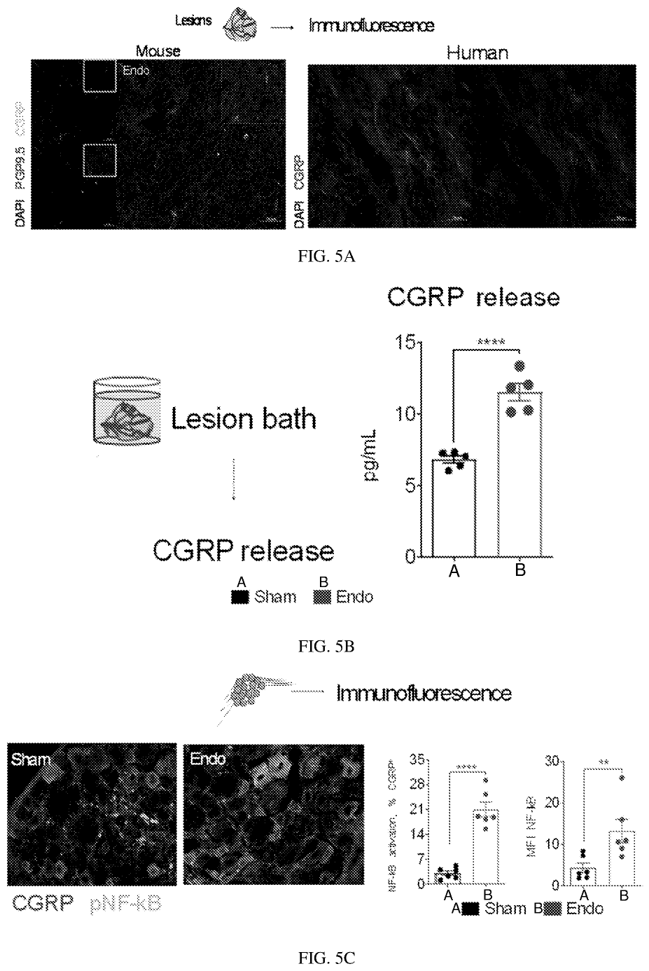

Mouse (56 days post induction (dpi)) and human endometriosis lesions were collected and stained for PGP9.5 (pan neuronal marker) and CGRP by immunofluorescence (FIG. 5A). Endometriosis lesions were collected at 56 dpi and the supernatant from the organ bath was collected to determine CGRP release by Enzyme immunoassay (EIA) (FIG. 5B). Results are expressed as mean±SEM of 5 wells per group per experiment, two independent experiments (Student's t-test, ****p<0.0001). DRG neurons from sham and endometriosis mice were dissected for determination of NF-kB activation in CGRP+ DRG neurons by immunofluorescence (FIG. 5C). Results shown are expressed as mean±SEM of 5 wells per group per experiment, two independent experiments (Student's t-test, ****p<0.0001; **p<0.02)

### Example 3: Endometriosis Lesion Activates TRPV1+ and TRPA1+ Nociceptors and Induce CGRP Release

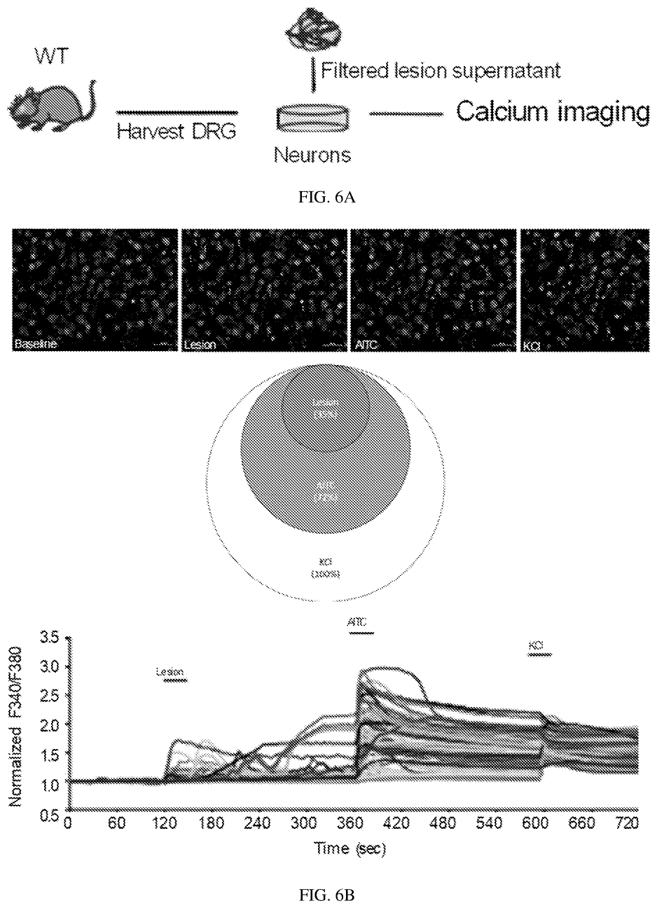

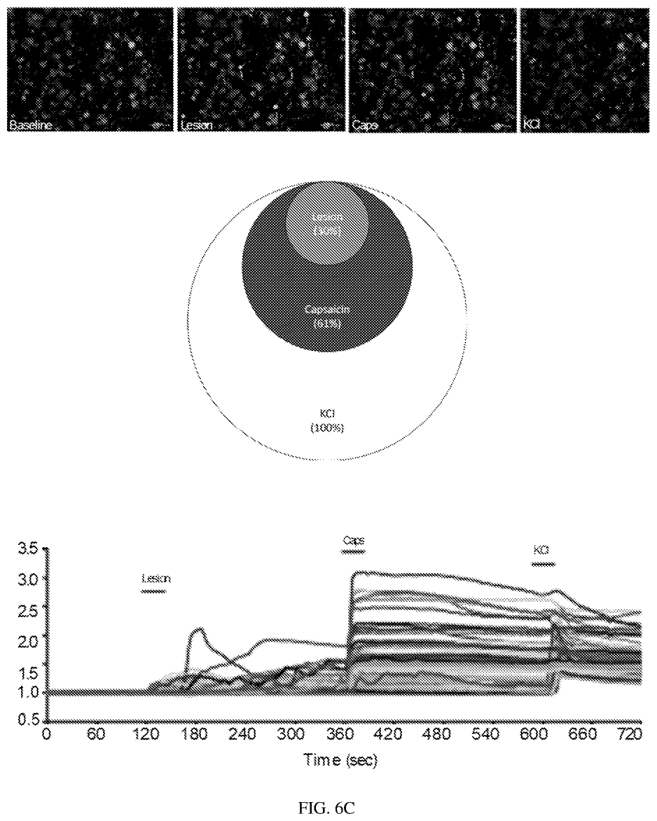

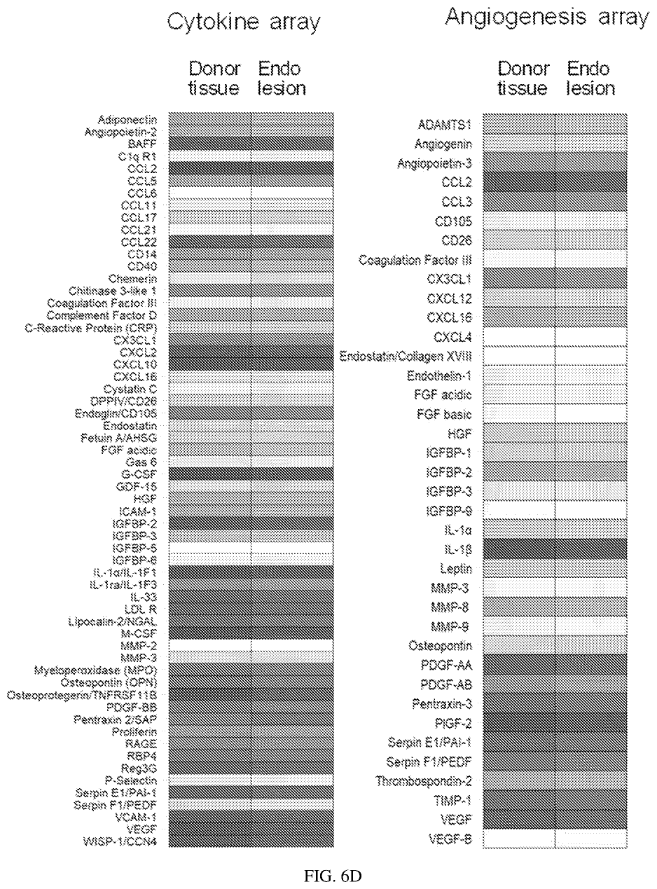

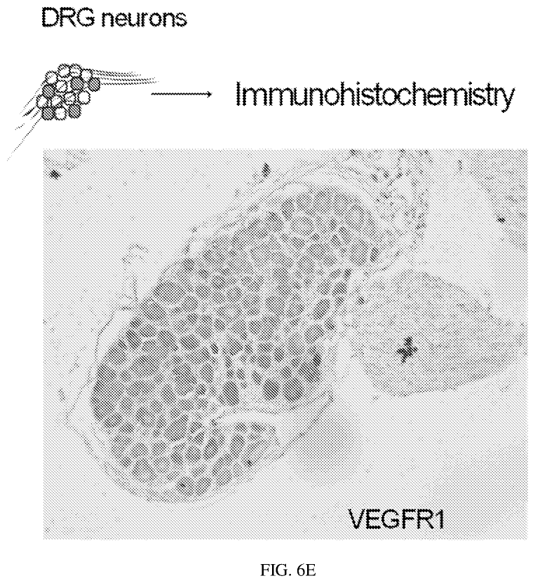

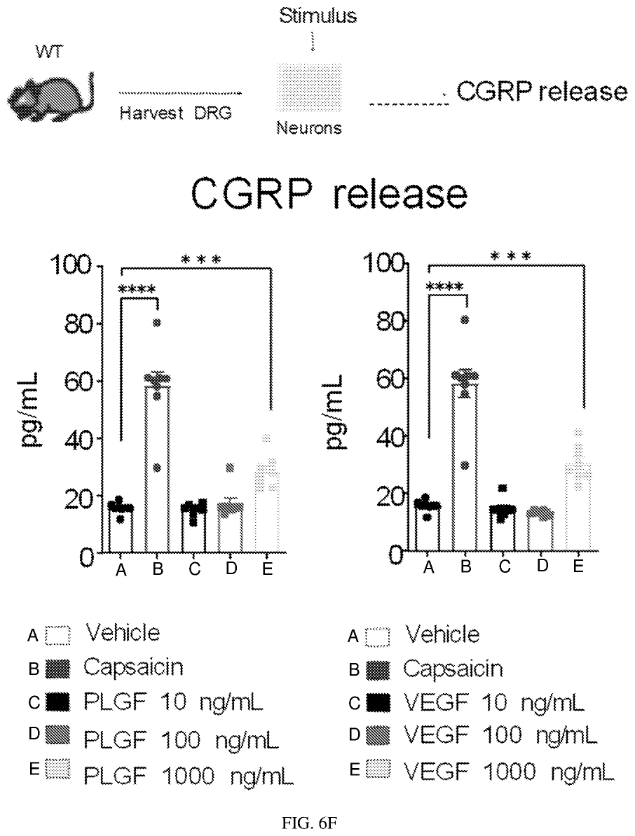

DRG neurons from naive mice were dissected for calcium imaging using Fura-2AM. Fluorescence ratio traces representing calcium influx into DRG cells from representative fields (FIGS. 6B-6C) throughout the 12 min of recording (FIGS. 6A-6C). Mean fluorescence ratio representing intracellular calcium concentration is shown at the baseline (zero-second mark) and following the stimulus with lesion supernatant (120s mark), TRPA1 agonist AITC or the TRPV1agonist capsaicin (360s mark) and KCI (600 seconds mark, activates all neurons). Endometriosis lesions were collected 56 dpi for determination of pro-inflammatory mediators using cytokine and angiogenesis array kits (FIG. 6D). Donor uterus was used as controls. DRG neurons from naive mice were dissected for determination of VEGFR1 receptor using IHC and determination of (c1) CGRP release by EIA (FIGS. 6E-6F). Supernatants were 1 h after stimuli to determine CGRP concentration by EIA. Results shown are expressed as mean±SEM of 6 wells per group per experiment, two independent experiments (one-way ANOVA followed by Tukey's post-test, ****p<0.0001); ***p<0.01).

### Example 4: TRPV1+ Nociceptors Control Endometriosis Lesions Establishment and Growth

TRPV1-cre mice were bred with rosa26(R26) DTA mice to produce mice do not express TRPV1. These mice were used in the following experiments to test whether TRPV1+ nociceptors control endometriosis lesions establishment and growth.

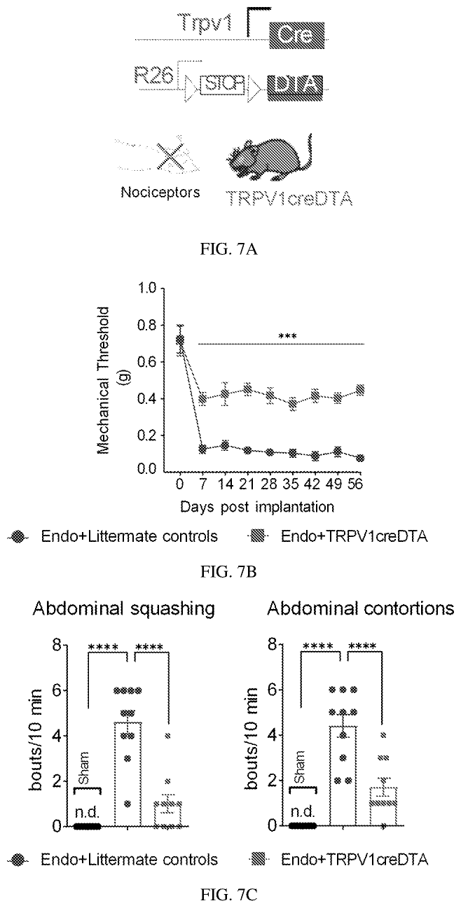

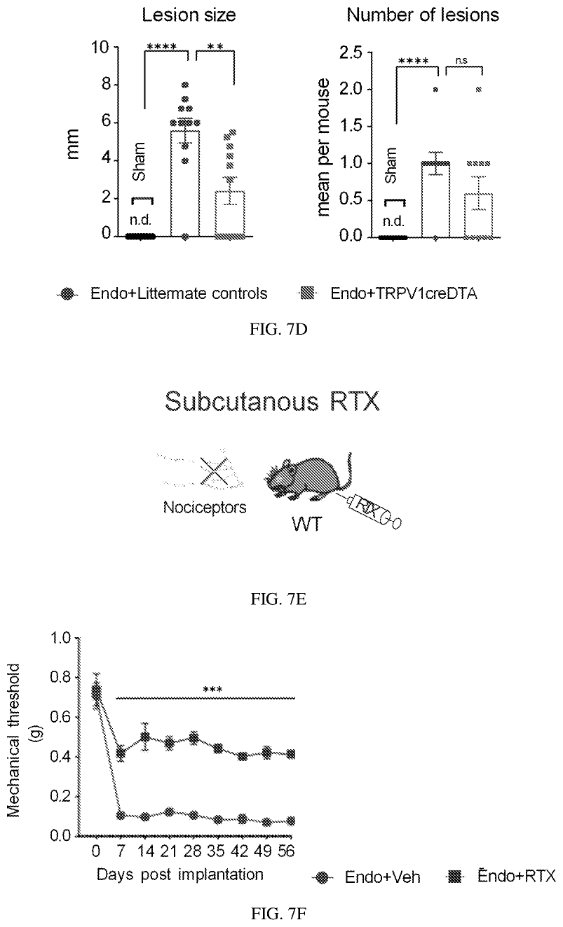

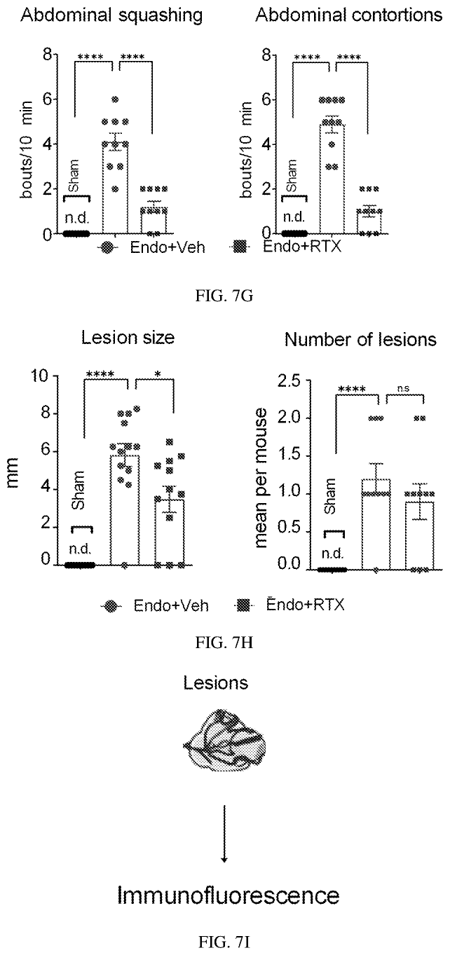

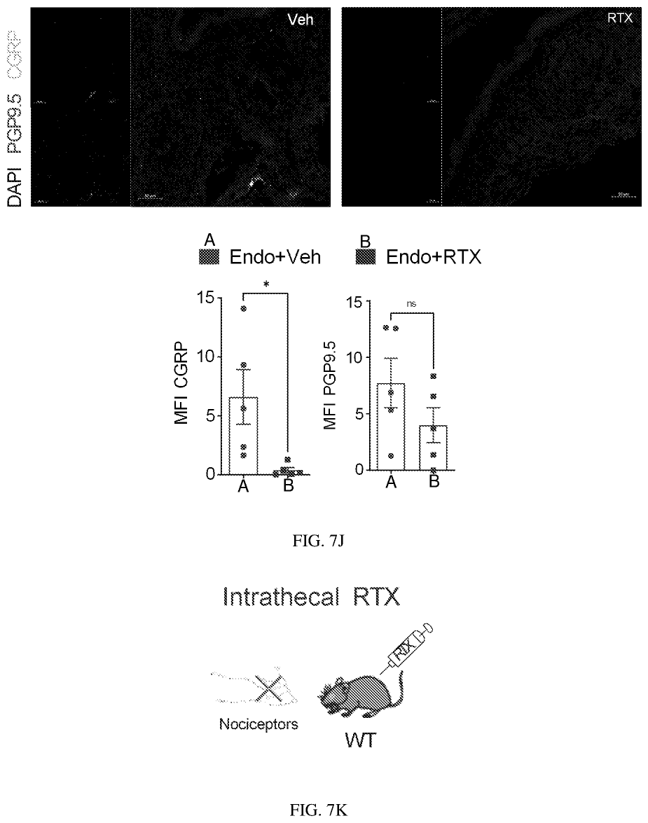

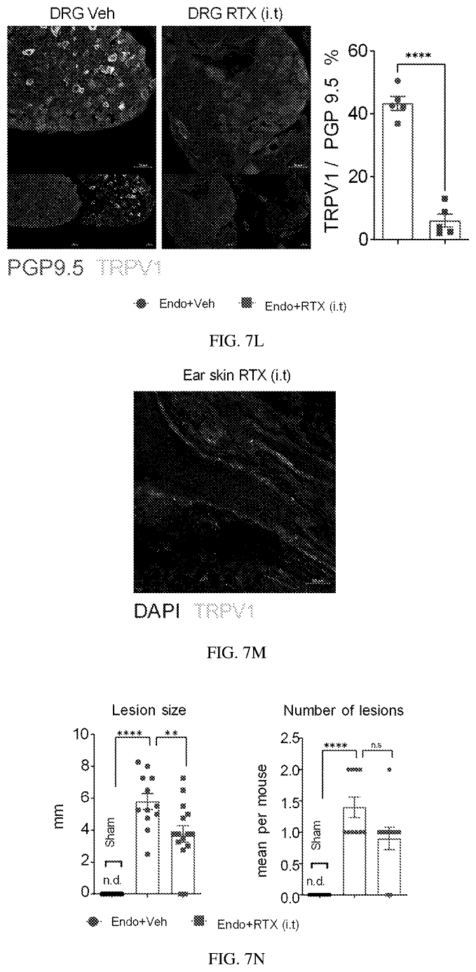

Abdominal mechanical hyperalgesia was measured before (zero) and after (7-56 dpi, weekly) using von Frey filaments (FIGS. 7B and 7F). Results are presented as mean±SEM of mechanical threshold, n=10 mice per group (two-way repeated-measures ANOVA followed by Tukey's post hoc, ***p<0.001). Spontaneous behaviors measurements were performed (FIGS. 7C and 7G.) For abdominal squashing, the number of times the mice pressed the lower abdominal region against the floor was quantified for 10 minutes. Sham mice did not display abdominal squashing. Abdominal contortions were quantified for 10 minutes by counting the number of contractions of the abdominal muscle together with stretching of hind limbs. Sham mice did not display abdominal contortions. Lesion size was determined in the remaining lesions by measuring height and weight (FIGS. 7D, 7H and 7N.) Sham mice did not show any lesion (n.d.=not detectable). Results shown are expressed as mean±SEM, n=10 (one-way ANOVA followed by Tukey's post hoc, ****p<0.0001). To confirm if RTX-induced (subcutaneous (sc) route) reduce CGRP fibers in the lesion, lesions were collected at 28 dpi for determination of CGRP staining by immunofluorescence (FIGS. 71-7J). Representative pictures are display in FIG. 7J. FIG. 7J also shows the quantification of CGRP and PGP9.5 staining upon RTX ablation (Student's t-test, *p<0.05). Confirmation of RTX-induced (intrathecal (it) route) in mouse DRG neurons or ears (28 dpi) stained for PFP9.5 and TRPV1 staining by immunofluorescence (one-way ANOVA followed by Tukey's post hoc, ****p<0.0001, **p<0.02) (FIGS. 7K-7N).

### Example 5: TRPV1+ Nociceptors Control F4/80+Ly6C+ Immune Cell Recruitment During Endometriosis

Macrophages are the main cells in the peritoneal cavity and are linked to the development of endometriosis. Markers of macrophages include F4/80 (marker of tissue macrophage), Ly6C (marker of monocyte), and F4/80+Ly6C+(marker of monocytes that were differentiated into macrophages).

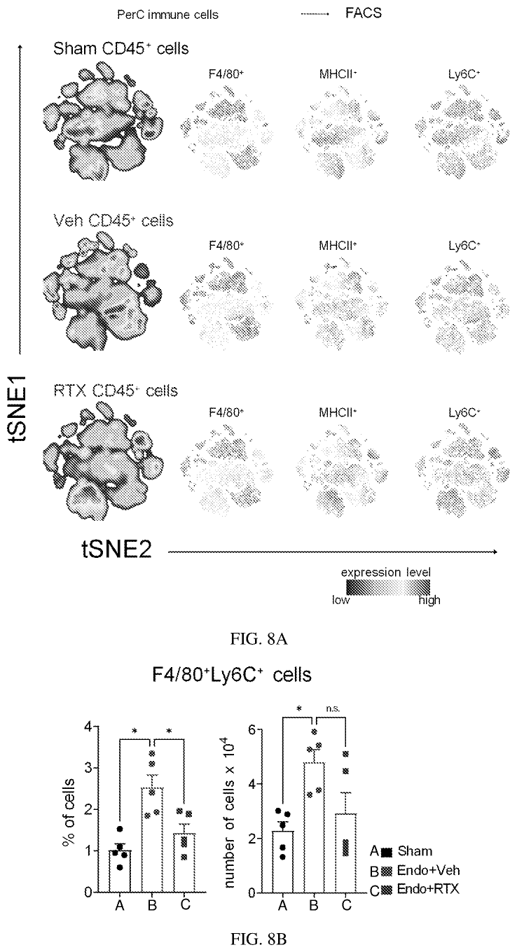

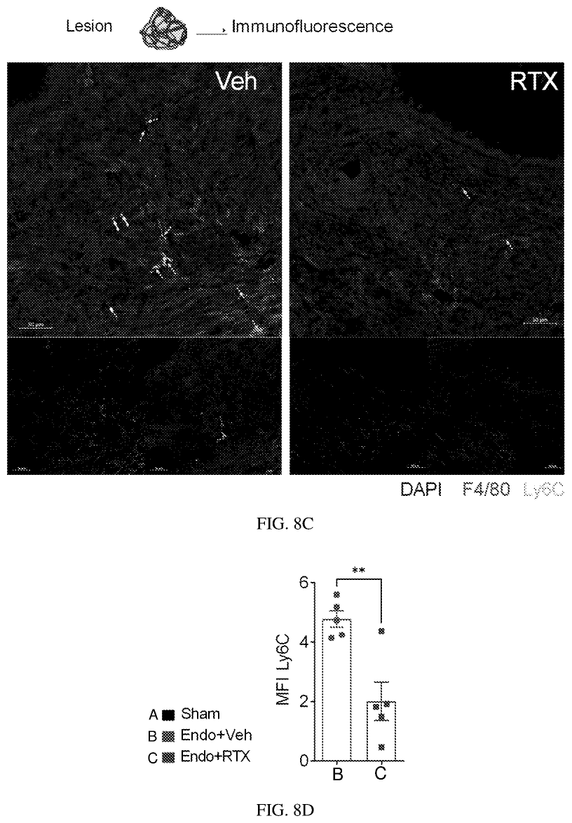

PerC wash (FIGS. 8A-8B) and lesions (FIGS. 8C-8D) were collected 28 dpi from sham, vehicle-, or RTX-treated mice for determination of monocyte recruitment by flow cytometry and immunofluorescence. Cluster analysis and t-distributed stochastic neighbour embedding (t-SNE) distribution of monocyte/macrophage subsets as represented by the markers CD45, F4/80, MHCII, and Ly6C (FIG. 8A). FIG. 8B displays quantification of F4/80+Ly6C+ cells performed by flow cytometry while FIG. 8D displays quantification by immunofluorescence. White arrows show F4/80+Ly6C+ cells while yellow arrows Ly6C+ only cells. Results shown are expressed as mean±SEM, n=5 (one-way ANOVA followed by Tukey's post hoc, **p<0.02, *p<0.05).

### Example 7: TRPV1+ Nociceptors Control F4/80+Ly6C+ Immune Cell Recruitment During Endometriosis

To deplete macrophages, two strategies were used. (1) Clodronate liposomes were employed that and (2) the CCR2-KO mice is a mouse line that lacks the receptor CCR2, which is widely expressed by monocytes.

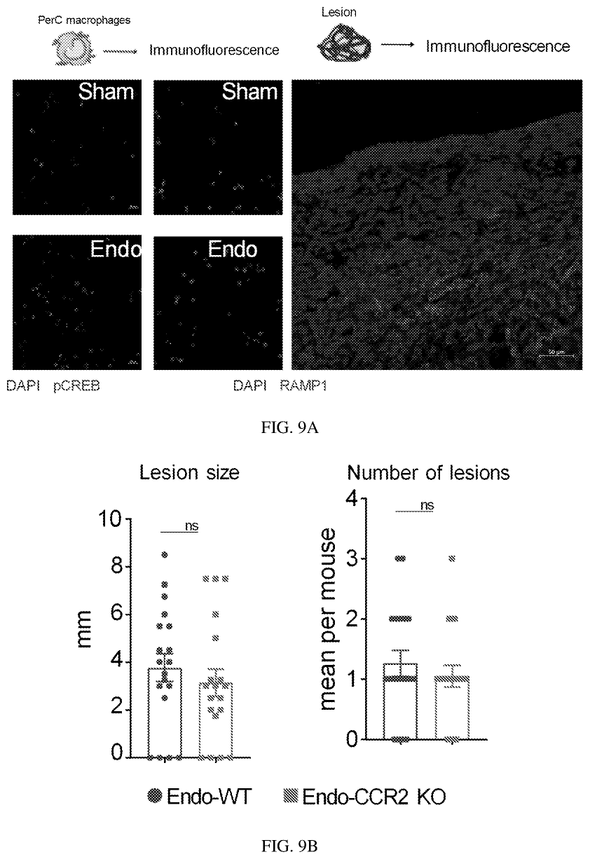

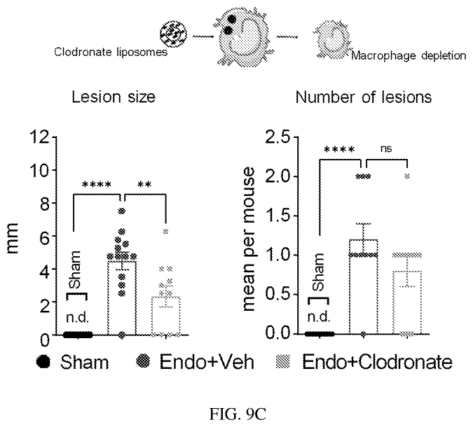

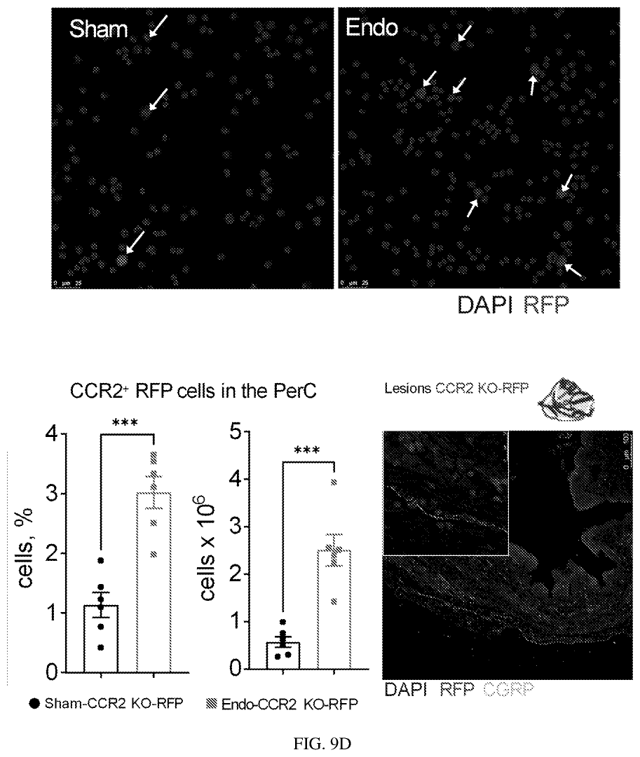

PerC wash and lesions were collected 56 dpi from sham or endometriosis mice for determination of pCREB activation or RAMP1 expression by immunofluorescence (FIG. 9A). To determine the impact of macrophage depletion during endometriosis, CCR2KO (FIG. 9B) or clodronate-depleted (FIG. 9C) mice were used. Lesion size was determined in the remaining lesions by measuring height and weight. Macrophages were plated onto tissue culture insert with mEEC plated on the bottom of the well. Stimulus with CGRP (100 nM) or vehicle was performed for 24 h and then mEEC were used to determine for cell proliferation assay using CyQuant kit. Treatment with Rimegepant (100 nM) or vehicle was performed 30 min before stimulus with CGRP. For the efferocytosis assay, macrophages were plated overnight onto a 96-well plate and stimulus with CGRP (100 nM) or vehicle was performed for 24 h. After that, etoposide-induced apoptotic 12z-eGFP were added to the macrophage culture and incubated for 1 h. Cells were washed 3 times with DPBS to remove non-engulfed 12z-eGFP and then and fluorescence intensity determined. Treatment with Rimegepant (100 nM) or vehicle was performed 30 min before stimulus with CGRP. PerC wash or lesions were collected 28 dpi in CCR2KO-RFP mice to determine the presence of CCR2+ monocytes in the PerC or lesion (FIG. 9D). Lesions were also stained with CGRP to determine the presence of these cells close to peptiderfgc nociceptors. Results shown are expressed as mean±SEM, n=5−12 (one-way ANOVA followed by Tukey's post hoc or Student's t-test, ****P<0.0001, p<0.001, **p<0.02, *p<0.05).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method for treating endometriosis, the method comprising administering to a subject in need thereof an effective amount of an agent that is sufficient to inhibit calcitonin gene-related peptide/receptor activity modifying protein 1 (CGRP/RAMP1) signaling in the subject.

2. A method for treating endometrioma, the method comprising administering to a subject in need thereof an effective amount of an agent that is sufficient to inhibit calcitonin gene-related peptide/receptor activity modifying protein 1 (CGRP/RAMP1) signaling in the subject.

3. The method of claim 1 or claim 2, wherein the agent comprises a small molecule or an antibody.

4. The method of claim 3, wherein the agent is a small molecule that binds to CGRP or RAMP1.

5. The method of claim 4, wherein the small molecule specifically binds to CGRP or RAMP1.

6. The method of claim 4 or claim 5, wherein the small molecule binds to CGRP, or RAMP1 on the cell surface of endometrial cells and/or macrophages of the subject.

7. The method of any one of claims 4-6, wherein the small molecule inhibits CGRP or RAMP1 signaling in endometrial cells and/or macrophages of the subject.

8. The method of claim 7, wherein the small molecule inhibits the signaling of CGRP or RAMP1 in endometrial cells and/or macrophages of the subject by at least 10%, at least 20%, at least 30%, at least 40%, at least 50%, at least 60%, at least 70%, at least 80%, at least 90%, at least 95%, or at least 99%.

9. The method of any one of claims 4-8, wherein the small molecule binds to RAMP1 is Rimegepant.

10. The method of any one of claims 4-8, wherein the small molecule binds to RAMP1 is Ubrogepant.

11. The method of claim 3, wherein the agent is an antibody that binds to CGRP or RAMP1.

12. The method of claim 11, wherein the antibody specifically binds to CGRP or RAMP1.

13. The method of claim 11 or claim 12, wherein the antibody binds to CGRP, or RAMP1 on the cell surface of endometrial cells and/or macrophages of the subject.

14. The method of any one of claims 11-13, wherein the antibody inhibits CGRP or RAMP1 signaling in endometrial cells and/or macrophages of the subject.

15. The method of claim 14, wherein the antibody inhibits the CGRP or RAMP1 signaling in endometrial cells and/or macrophages of the subject by at least 10%, at least 20%, at least 30%, at least 40%, at least 50%, at least 60%, at least 70%, at least 80%, at least 90%, at least 95%, or at least 99%.

16. The method of any one of claims 11-15, wherein the antibody binds to CGRP and is Fremanezumab, Eptinezumab or Galcanezumab.

17. The method of any one of claims 11-15, wherein the antibody binds to RAMP1 and is Erenumab.

18. The method of any one of claims 1-17, wherein method comprises administering an effective amount of the agent that is sufficient to inhibit CGRP/RAMP1 signaling in endometrial cells and/or macrophages of the subject.

19. The method of claim 18, wherein the endometrial cells are located in endometrial lesions of the subject.

20. The method of claim 18, wherein the macrophages are peritoneal macrophages or macrophages located in reproductive tissue of the subject.

21. The method of any one of claims 1-20, wherein the method results in a reduction of inflammation in endometrial lesions of the subject.

22. The method of any one of claims 1-21, wherein the method results in a reduction of nociception in endometrial lesions of the subject.

23. The method of any one of claims 1-22, wherein the method results in an enhancement of efferocytosis in macrophages of the subject.

24. The method of claim 23, wherein the macrophages are peritoneal macrophages or macrophages located in reproductive tissue of the subject.

25. The method of any one of claims 1-24, wherein the subject is a human patient.

26. The method of any one of claims 1-25, wherein the administration is oral or via injection.

27. The method of any one of claims 1-26, wherein the administration is systemic.

28. The method of any one of claims 1-27, wherein the administration is local.

29. The method of any one of claims 1-28, wherein the agent is administered to endometrial cells of the subject.

30. The method of claim 29, wherein the agent is administered to endometrial lesions of the subject.

31. The method of any one of claims 1-30, wherein the agent is administered more than once.
